(19) United States
(12) Patent Application Publication
(10) Pub. No.: US 2018/0064401 A1
(43) Pub. Date: Mar. 8, 2018

(54) RECONFIGURABLE POINT-OF-EVENT PUSH DIAGNOSTIC SYSTEM AND METHOD

(71) Applicant: Smart Monitor Corp, San Jose, CA (US)

(72) Inventor: Anooradah Nathan, San Jose, CA (US)

(21) Appl. No.: 15/697,395

(22) Filed: Sep. 6, 2017

Related U.S. Application Data
(60) Provisional application No. 62/384,827, filed on Sep. 8, 2016.

(51) Int. Cl. A61B 5/00; A61B 5/0205; A61B 5/11

(52) U.S. Cl. CPC A61B 5/7275; A61B 5/4815; A61B 5/02055; A61B 5/0015; A61B 5/11; A61B 5/024; A61B 5/6801; A61B 5/4266; A61B 5/4845; A61B 5/7264; A61B 2562/0219; A61B 5/7465

(57) ABSTRACT

The present invention describes methods and systems to enable a concerned party to continuously monitor the progression of a medical condition in one or more patients and push event specific physical and or cognitive diagnostic to the patient in real time to effectively track and assessing post-episodic recovery for a variety of diseases states. The progression of the medical condition is determined by processing sensor data obtained from one or more physiological and/or motion sensors and survey data obtained from the patients. Further, environmental data such as air quality, temperature and humidity may also be used along with the sensor data and the survey data to monitor/track the progression of the medical condition.

### CROSS REFERENCE TO RELATED APPLICATIONS

This application is a claims the priority benefit of U.S. provisional applications 62/384,827, filed on Sep. 8, 2016 inventor Anooradah Nathan, the complete contents of the applications are incorporated in its entirety herein by reference.

### BACKGROUND OF THE INVENTION

### Field of the Invention

The present invention generally relates to a reconfigurable point-of-event diagnostic system, particularly for monitoring patients in real time and pushing event-specific physical and cognitive diagnostic. The invention discusses system and method to monitor patients in real time to determine possible onset of any neurological event based on static risk profile and push an event-specific cognitive and or physical diagnostic.

### Description of Related Art

A neurological disorder causes structural, biochemical or electrical abnormalities in the brain, spinal cord or other nerves can result in a range of symptoms. Examples of symptoms include paralysis, muscle weakness, poor coordination, loss of sensation, seizures, confusion, pain and altered levels of consciousness. There are many recognized neurological disorders, some relatively common, but many rare. They may be assessed by neurological examination, and studied and treated within the specialties of neurology and clinical neuropsychology.

The World Health Organization estimated that neurological disorders and their sequelae (direct consequences) affect as many as one billion people worldwide, and affect people in all countries, irrespective of age, sex, education, religion or income. Interventions for neurological disorders include preventative measures, lifestyle changes, physiotherapy or other therapy, neurorehabilitation, pain management, medication, or operations performed by neurosurgeons.

Despite the availability of highly effective, low cost medication, many patients suffering from various neurological disorders, such as epilepsy, stroke etc., go untreated at the exact time of the occurrence of an episode. In the past, such a diagnosis/treatment has been performed by doctors in hospital settings. It is typically performed using detailed medical examinations. EKG and other medical tests.

Therefore, there is a need of a method and/or system that can monitor patients and detect possible onset of neurological events in patients, even when they are not in a hospital. Additionally, diagnostic tests could be administered to the patient during and, or after the onset of an occurrence of an episodic event for a better assessment of patient. The present invention addresses these issues and provides a better framework for monitoring and administering real-time diagnostic tests thus, reducing deaths and saving and monitoring lives.

### SUMMARY OF THE INVENTION

In an embodiment of the invention, the reconfigurable point-of-event diagnostic system comprises of a body-worn factor configured for capturing any one of a physical, physiological and, or motion characteristic set of parameters for more advanced analytics than prior art in this field. This invention integrates both physical sensors and biological sensors to join into a new level of analysis and algorithms explained here. The invention is a notable step forward in creating more intelligent and complex analysis of multi-sensor applications to analyze the human body.

More specifically, the present invention relates to a system to monitor a plurality of patients to detect possible onset of a neurological condition and/or other similar medical conditions. In an embodiment of the present invention the reconfigurable point-of-event diagnostic includes capturing at least the first set of physiological, physical and, or motion parameters of a patient and based on at least the first set of parameters, compare to a static risk profile, detect a threshold-grade deviation therefrom to deduce a neurological event type. Further yet, in an embodiment of the invention based on the neurological event type, push at least one of an event-specific cognitive and, or physical diagnostic request further validating the neurological event by the validation module based on the results of the diagnostic. Additionally, in an embodiment of the invention, the information of the validated neurological event is pushed to any one of a designated recipient. Further yet, in an embodiment of the invention, the motion characteristics of the patient correspond to at least one of activity related characteristics or sleep related characteristics of the patient.

The motion characteristics of the patient correspond to at least one of activity related characteristics and sleep related characteristics of the patient. Examples of activity related characteristics of the patient include, but are not limited to, maximum value of acceleration, minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

Going further, sleep related characteristics of the patient are indicative of at least one of the group comprising sleep time, number of times awake, duration of sound sleep, duration of light sleep and awake time. The physiological characteristics of the patient are one or more of group comprising heart rate, pulse rate, respiratory rate and body temperature. Additionally, in an embodiment a set of parameters corresponding to the physical characteristics is based on at least one of, age, gender, race, medical history, heart rate, medication history, blood pressure, sweat, duration, number of episodes and history of disease, family history, fatigue, walking/running/movement related impairment, bladder dysfunction, vision and speech impairments, food habits, smoking, alcohol and drug intake.

In some embodiments, the invention will acquire a first set of parameters from the various body physical/geometric sensors on the body worn device, such as acceleration sensors (accelerometers), angle sensors (gyros), location sensor (GPS), directional sensor (compass or magnetometers). Additionally, physiological sensors (such as body temperature, heart rate/pulse etc.) can also be used. Further yet, in another embodiment of the invention, a second set of contextual parameters of the patient is captured from a plurality of devices.

The method and system further includes validating the neurological event by the validation module based on the response to at least one of the event-specific physical and, or cognitive diagnostic and pushing the generated information in the form of a report to a concerned party such as a healthcare provider, a hospital, a health monitoring service, a doctor, a physician, a clinician, a caregiver and a social service.

The invention will typically employ one or more processors (e.g. compute processors such as microprocessors, and the like) and several types of analytics algorithms to analyze the characteristics of various daily normal activities. These normal activities can include walking, running, talking, sleeping and the like. The invention can use data from these various sensors and devices to establish a baseline of what is “normal” for these types of activities. These baselines may be established across groups of individuals and/or customized for each individual. These baselines may also be adjusted for different situations.

### BRIEF DESCRIPTION OF DRAWINGS

FIG. 1 describes an exemplary embodiment of the network.

FIG. 2 illustrates an exemplary interaction flow in which various embodiments of the disclosure can be practiced.

FIG. 3 illustrates an exemplary system in which various embodiments of the disclosure can be practiced.

FIG. 4 illustrates an exemplary process flow according to an embodiment of the invention.

FIG. 5 show a user interface according to an exemplary embodiment of the invention.

FIG. 6 depicts a method flowchart for validation of neurological events.

### DETAILED DESCRIPTION OF DRAWINGS

The present invention will now be described more fully with reference to the accompanying drawings, in which embodiments of the invention are shown. However, this disclosure should not be construed as limited to the embodiments set forth herein. Rather, these embodiments are provided so that this disclosure will be thorough and complete, and will fully convey the scope of the disclosure to those skilled in the art. Like numbers refer to like elements throughout.

In the following description, for purposes of explanation, numerous specific details are set forth in order to provide a thorough understanding of the invention. It will be apparent, however, to one skilled in the art that the invention can be practiced without these specific details.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

### Overview:

The primary purpose of the disclosure is to detect a neurological event type and push at least on of an event-specific physical and or cognitive diagnostic to one or more patients in real time. Typically, the progression of the medical condition is determined by processing sensor data obtained from one or more physiological and/or motion sensors and survey data obtained from the patients. Further, environmental data such as air quality, temperature and humidity may also be used along with the sensor data and the survey data to monitor/track the progression of the medical condition.

The present disclosure focuses on detecting a threshold-grade deviation therefrom to deduce a neurological event type and further, pushing event-specific physical and or diagnostic to the patient in real time. However, for a person skilled in the art it is understood these examples are just for understanding purposes and the disclosure can be implemented for objects other than medical condition monitoring for example fitness level monitoring.

Examples of the medical condition include, but are not limited to, Multiple Sclerosis (MS), Primary Progressive Multiple Sclerosis (PPMS), Huntington's disease (Chorea), Epilepsy & Seizures, Parkinson's disease, Post Stroke conditions, Tobacco use related conditions, Asthma, Cancer, Arthritis, Chronic Obstructive Pulmonary Disease (COPD), Diabetes, heart disease, Obesity, Osteoporosis, Alzheimer's disease, Reflex Sympathetic Dystrophy (RSD) Syndrome, Pruritus and Chronic kidney disease (CKD). Going forward in this disclosure, the present invention will be described taking the example of Multiple Sclerosis. However, for a person skilled in the art it is understood that this example is just for understanding purposes and the disclosure can be implemented for other medical conditions that may result in impairment with respect to motion in the patients.

### Exemplary Environment

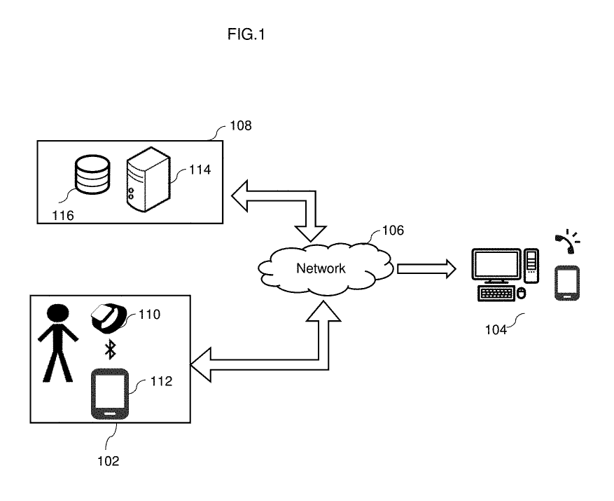

FIG. 1 illustrates an exemplary environment 100 in which various embodiments of the present invention can be practiced. The environment 100 includes patient premises 102, a concerned party 104 and a processing unit 108. The patient premises 102, the concerned party 104 and the processing unit 108 are communicatively coupled through a network 106. Typically, the processing unit 108 enables the concerned party 104 to continuously monitor the symptoms of a medical condition such as Multiple Sclerosis (MS) in the patient, located at the patient premises 102.

Examples of the concerned party 104 include, but are not limited to, a healthcare provider, a hospital, a health monitoring service, a doctor, a physician, a clinician, a caregiver and a social service. The processing unit 108 may either be operated by the concerned party 104 or a third-party. Examples of the third party include, but are not limited to, a service provider that specializes in continuously collecting medical data from patients and distributing the medical data to a plurality of concerned parties. Typically, the processing unit 108 includes a processor 114 and a medical information database 116.

The patient premises 102 is a place, the patient is located at, other than a hospital or any other similar medical institution/setting. To enable continuous monitoring, the patient uses a body worn device 110 and a mobile communication device 112. The body worn device 102 is typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors. In an embodiment of the present invention, the data captured by the one or more sensors is sent to the processing unit 108 through the network 106.

Typically, the body worn device 102 is worn on one or more body parts of the patient, such as wrist, waist, neck, arm, leg, abdomen, chest, thigh, head, ear and fingers. Further, the body worn device 102 may be a wristband, a watch, an armband, a necklace, a headband, an earring, a waist belt and a ring.

The mobile communication device 114 is a portable device that has the capability of communicating over the network 106, presenting periodic surveys to the patient and receiving response from the patient on the periodic surveys. Examples of the mobile communication device 114 include, but are not limited to, a smartphone, a tablet, a personal digital assistant (PDA) and a mobile phone.

In an embodiment of the present invention, the data captured by the one or more sensors of the body worn device 110 is first sent to the mobile communication device 112 and thereby, sent to the processing unit 108 over the network 106. The body worn device 110 communicates with the mobile communication device 112 over a short range wireless communication medium. Examples of the short range wireless communication medium include Bluetooth, ZigBee, Infrared, Near Field Communication (NFC) and Radio-frequency identification (RFID).

The network 106 may be any suitable wired network, wireless network, a combination of these or any other conventional network, without limiting the scope of the present invention. Few examples may include a LAN or wireless LAN connection, an Internet connection, a point-to-point connection, or other network connection and combinations thereof. The network 106 may be any other type of network that is capable of transmitting or receiving data to/from host computers, personal devices, telephones, video/image capturing devices, video/image servers, or any other electronic devices. Further, the network 106 is capable of transmitting/sending data between the mentioned devices. Additionally, the network 106 may be a local, regional, or global communication network, for example, an enterprise telecommunication network, the Internet, a global mobile communication network, or any combination of similar networks. The network 106 may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. The network 106 may include any software, hardware, or computer applications that can provide a medium to exchange signals or data in any of the formats known in the art, related art, or developed later.

In an embodiment of the present invention, the processing unit 108 receives the sensor data from the body worn device 110 and response on the periodic surveys from the patient. Thereby, the processing unit 108 correlates the sensor data with the response on periodic surveys, generates reports corresponding to the symptoms of medical condition in the patient and sends the reports and other relevant data to the concerned party 104. These reports enable the concerned party 104 to track/monitor the progression of the medical condition in the patient.

In an embodiment of the present invention, the concerned party 104 is enabled to view the reports, as generated by the processing unit 108 using one or more devices selected from the group comprising a smartphone, a computer, a laptop, a tablet, a personal digital assistant (PDA) and a mobile phone.

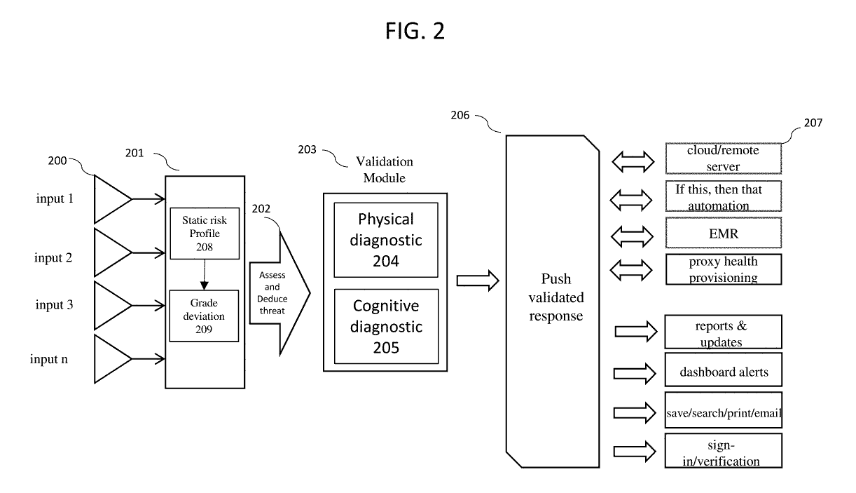

FIG. 2., illustrates an exemplary interaction flow of a reconfigurable point-of-event diagnostic for capturing, detecting and assessing a neurological event 202 from the multiple device inputs 200 and validating an event-specific physical 204 and or cognitive 205 diagnostic by the validation module 203 and pushing the information of the validated neurological event 206 to any one of a designated recipient. In a preferred embodiment of the invention, a reconfigurable point-of-event diagnostic system comprises of a body-worn device 200 is configured for capturing at least one of a physical, physiological and, or motion parameter, a network interface, a processor, a validation module 203, a non-transitory storage element coupled to the processor with encoded instructions stored in the non-transitory storage element, wherein the encoded instructions when implemented by the processor configure the diagnostic system to capture at least one of the physiological, physical and, or motion parameter of a patient and compare to a static risk profile 208. Further yet, in a preferred embodiment of the invention, a threshold-grade deviation 209 is detected to therefrom deduce a neurological event type and based on the neurological event type, at least one of an event-specific cognitive 205 and, or physical 204 diagnostic is pushed. Additionally, in a preferred embodiment of the invention, based on the response to at least one event-specific cognitive 205 and, or physical 204 diagnostic, validation of the neurological event by the validation module 203 is performed and the information of the validated neurological event is pushed 206 to any one of a designated recipient.

Once the device input 200—however disparate—is aggregated, reconfigurable point-of-event diagnostic system captures at least one set of the physiological, physical and or motion parameters of a patient, it compares it to a static risk profile 208. If a discrepancy is above a pre-defined threshold and or if the system detects a threshold-grade deviation 209, then the system, assesses 202 the potential threat, alerts the patient and or designated recipient and pushes an event-specific physical 204 and or cognitive 205 diagnostic to the patient. Based on the response to at least one physical and or diagnostic, the validation module validates the neurological event and alerts the designated recipient. In another embodiment, in addition to alerting the patient, the reconfigurable point-of-event system may be configured to provide at least one automated response to the assessed and alerted patient. Further yet, the patient alert may be at least one of, text, email, vibration with or without audible notification, visual display, and, or a color-coded or blinking notification.

In a preferred embodiment, device data input 200 may encompass the sensor-captured raw data input or transduced and processed data input from the body worn device that is the subject of the supra device and method claims. Device input 200 may also encompass the sensor-captured raw data input or transduced and processed data input from any other device associated with the patient/user. Examples may be devices worn, mobile devices, and, or fixed-access devices, such as Internet-of-Things devices (e.g. smart thermostat, home automation consoles, etc.). The plurality of device inputs provides additional input for aggregation and behavior profiling, thus layering the behavior profile with additional context for generating a higher fidelity of predictive analytics. Alternatively, the data input 200 may be from at least two disparate devices—capturing and processing non-overlapping motion or behavior parameters. In yet other embodiments, the data input 200 may be from at least two disparate devices—capturing and processing overlapping motion or behavior parameters. For instance, the reconfigurable system may employ a 6-axis accelerometer data input 200 of the body worn device and the 3-axis accelerometer data input 200 of an additionally worn fitness tracker and stack these motion metrics to generate a composite gait profile. Conversely, the data input 200 may be from at least two disparate devices capturing non-overlapping metrics, such as accelerometer data from the body worn device and gyroscopic data from a fitness tracker to inform the composite gait profile.

In an alternative embodiment of the invention, these multiple and heterogenous data inputs 200 may converge in an integration layer (not shown). The integration layer may further manage the data packets—of varying format—and collate into discrete bundles of packets/formats. In other embodiments, the integration layer may serve as a data format converter, converting the plurality of data formats—from disparate devices—into a universally recognized format. In yet other embodiments, the plurality of data inputs 200 and formats converge into the interaction policy layer for any one of collating the disparate data formats from a multitude of devices; and, or, converting the disparate data formats into a universally recognized data format; and aggregating the bundled and, or converted data inputs for configuring a composite static profile 208.

Devices—however disparate—including the body worn device, can communicate with integration layer wirelessly via Bluetooth—or any other short-range communication protocol—interfacing with any one of a mobile phone, Wi-Fi router and Wide Area Network access. The care-flow controller aggregates a first set of parameters from the body worn device and a second set of parameters from at least one of, a mobile communication device, wearable device, smartphone, tablet, personal digital assistant (PDA) and Internet of Things device.

The static risk profile 208 or any other reference profile (not shown), take into account complete device behavior. Device behavior includes not only data output informed by patient/user behavior, but also data output informed by network and device technical characteristics. Such technical characteristics may consider network traffic, bandwidth, network bottlenecks, network malfunctioning, device malfunctioning, sensor data acquisition fidelity, signal transduction, latency, patient feedback, etc. By taking in such device and network technical characteristics, the system may be able to make a secondary assessment of a discrepancy threshold and rule out device or network malfunctioning—verifying that the threshold discrepancy is due to a patient threat. In some embodiments, the event-specific physical and or cognitive diagnostic is pushed or the alert of a patient threat and, or automated response is triggered only after the device and, or network anomaly is ruled out after the discrepancy threshold is reached.

Still in reference to FIG. 2, a threshold discrepancy of an event may detect a threat, whereby the threshold discrepancy is determined by machine learning algorithms. Machine learning algorithms may be employed to inform a threshold discrepancy rater to determine whether a discrepancy threshold has been reached. Further yet, a machine learning algorithm may be employed to inform upstream processes. Additionally, in the point-of-event diagnostic system, the aggregation of the set of parameters occurs from at least one of, a mobile communication device, wearable device, smartphone, tablet, personal digital assistant (PDA) and Internet of Things device.

Further yet, in another embodiment, the reconfigurable point-of-event diagnostic system may further comprise integration with any one of a third-party application via an Application Program Interface (API) 207. This allows for 3^{rd} party database integration, such as Electronic Medical Records (EMR), health monitoring, proxy health provisioning, remote server and, or a cloud based server for other 207 downstream analytics and provisioning. Additionally, the completed automated responses may be saved onto a remote cloud based server for easy access for data acquisition and archival analytics for future use.

In another embodiment of the invention, point-of-event diagnostic system may allow for easy saving, searching, printing, and sharing of completed automated response information with authorized participants. Additionally, the care-flow controller may allow for non-API applications, for example, building reports and updates, create dashboard alerts as well as sign in/verifications 207. Alternatively, sharing may be possible with less discrimination based on select privacy filters.

In another embodiment of the invention, at least one event-specific diagnostic triggers at least one action controlled by a “if this, then that” script manager in the point-of-event diagnostic system. Additionally, the “if this, then that” script manager is further embedded with an “and, or” trigger or action operators, allowing increased triggers or actions in a command set.

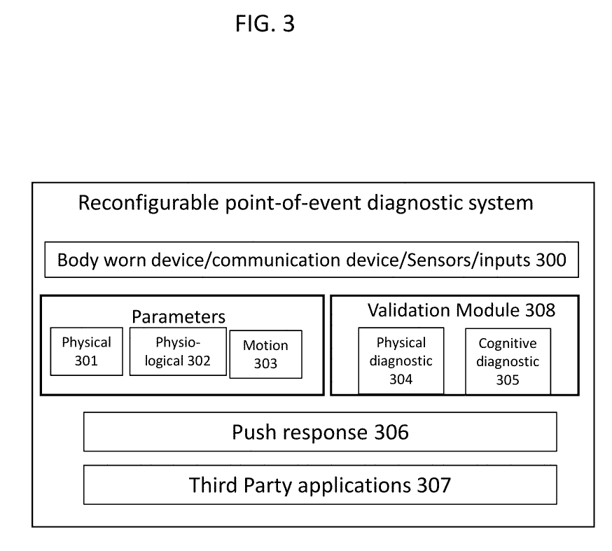

FIG. 3 illustrates an exemplary system in which various embodiments of the disclosure can be practiced. In a preferred embodiment of the invention, the inputs 300 captures any one of the physical 301, physiological 302 and or motion 303 parameters and processes input from anyone of, multiple sensors 300 on the body worn device, a plurality of devices, patients, caregivers, doctors or concerned, compares it to a static risk profile and is processed for detecting a threshold-grade deviation therefrom to deduce a neurological event type. Based on the neurological event type, an event-specific physical 304 and or cognitive 305 diagnostic is pushed to the patient in real time. Further yet, in an embodiment of the invention, based on the response to at least one even-specific cognitive 305 and or physical 304 diagnostic, the response is validated by a validation module 308 and subsequently, the response is pushed 306 in real time informing the patient, caregiver, doctor or a concerned party of an imminent threat. Further yet, in another embodiment, the reconfigurable point-of-event diagnostic system may further comprise integration with any one of a third-party application 307 via an Application Program Interface (API) 207. This allows for 3^{rd} party database integration, such as Electronic Medical Records (EMR), health monitoring, proxy health provisioning, remote server and, or a cloud based server for other 308 downstream analytics and provisioning. Additionally, the completed automated responses may be saved onto a remote cloud based server for easy access for data acquisition and archival analytics for future use.

Further yet, in an embodiment of the invention, the inputs 300 may be motion characteristics corresponding to at least one of, physical 301 activity, physiological 302 and sleep related characteristics of the patient. Additionally, the physical 301 and physiological 302 activities may have a set of parameters corresponding to activity related characteristics of the patient to be at least one of, but not limited to, maximum/minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time. Moreover, the sleep related characteristics of the patient may be indicative of at least one of, duration of sleep time, number of times awake, sound sleep, light sleep and awake time.

Additionally, environmental conditions may affect patient activity. The environmental conditions can be at least one of, but not limited to, wind velocity, temperature, humidity, aridness, light, darkness, noise pollution, exposure to UV, airborne pollution and radioactivity. Further yet, data generated from a set of parameters corresponding to at least one of, but not limited to, patient reported symptoms and side effects, periodic surveys may be used to generate a behavioral profile of a patient. The data generated may in any one of, but not limited to, audio, video or an image input and further, implemented on at least one of, but not limited to a mobile communication device, body worn device, wearable device, tablet and or IoT.

Further yet, in another embodiment of the invention, the point-of-event diagnostic system further comprises of a second set of parameters which may be provided by the patient using at least one of a mobile communication device and, or any other device. Additionally, in an embodiment of the invention, a set of parameters corresponding to the physical characteristics is based on at least one of, age, gender, race, medical history, heart rate, medication history, blood pressure, sweat, duration, number of episodes and history of disease, family history, fatigue, walking/running/movement related impairment, bladder dysfunction, vision and speech impairments, food habits, smoking, alcohol and drug intake. In an embodiment of the invention, the point-of-event diagnostic system further comprises of constructing a static risk profile based on at least one of, the set of physical, physiological or motion parameters and, or a plurality of combinations from a finite set of the set of parameters. Additionally, the point-of-event diagnostic system may comprise establishing a personal static risk profile of the patient based on at least one of the set of parameters. Moreover, the point-of-event diagnostic system may comprise detection a deviation from the personal static risk profile of the patient using machine learning algorithm.

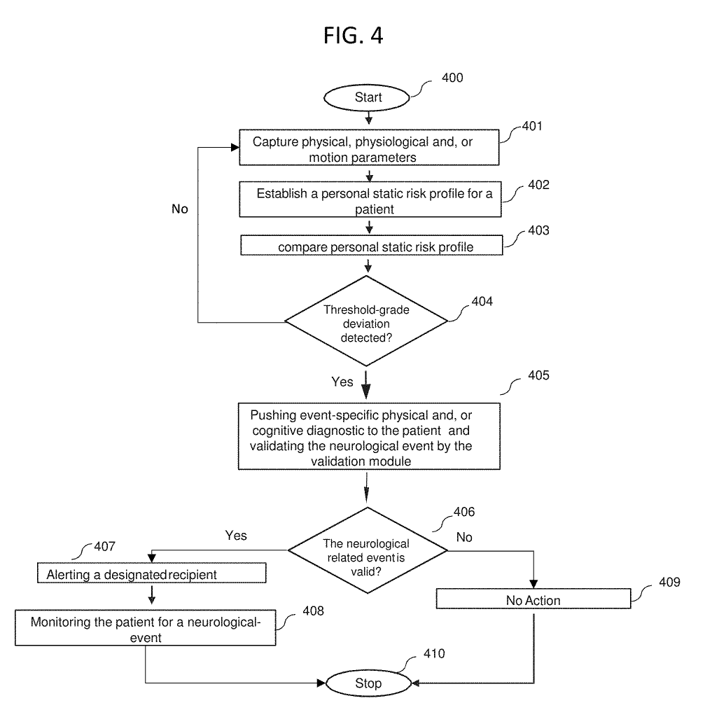

FIG. 4 illustrates an exemplary process flow according to an embodiment of the invention.

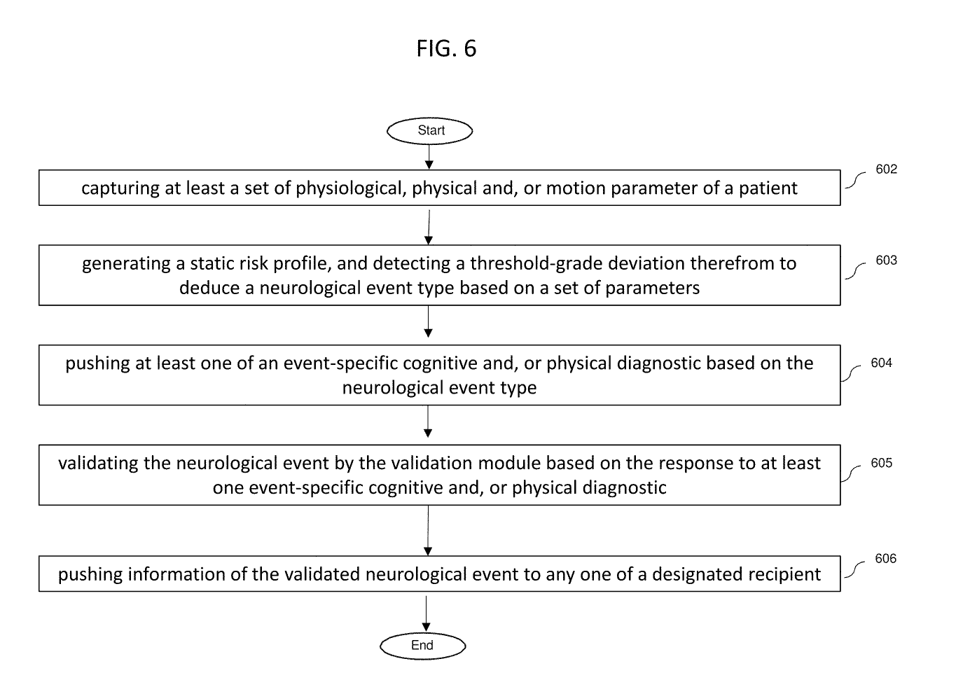

As illustrated in FIG. 6, a plurality of body-worn, wearable devices or any other device on a patient or user begins 400 to captures at least one physical, physiological and or motion parameter 401. A Wi-Fi connection is automatically established between at least one of, body-worn, wearable device or any other device and the server (not shown). A personal static risk profile for a patient is established 402 based on at least one set of parameters and compared to a static risk profile 403 to detect a threshold-grade deviation therefrom to deduce a neurological event type 404. If a threshold-grade deviation is detected then an even-specific physical and or cognitive diagnostic is pushed to the patient 405. Alternatively, if no threshold-grade deviation is detected, then capturing of at least one of the physical, physiological and or motion parameters is continued. Further yet, a validation module validates the neurological event 405 based on the response to at least one event-specific physical and or cognitive diagnostic. If a neurological event is validated 406, then a designated recipient is alerted 407 and the patient is further monitored for a relapse of an episode 408. Alternatively, if the neurological event is not validated, then no further action is required 409 and the process is completed 410.

In an embodiment of the invention, the point-of-event diagnostic system, wherein the event-specific physical and, or cognitive diagnostic may be validated by the validation module obtaining data from any one of, body worn sensors, sensor based devices used by patients, mobile communication device, smartphone, tablet, personal digital assistant (PDA) and, or Internet of Things device. Further yet, the point-of-event diagnostic system, may further comprise of a second set of parameters which is provided by the patient using at least one of a mobile communication device and or any other device. This may also contribute in calculating a risk profile of a patient which is dynamic or the dynamic risk profile.

Further yet, in an embodiment, a static risk factor value is calculated for the patient that defines the likelihood of the patient having a neurological event. The static risk factor value is calculated based on one or more of the static parameters described above. Typically, the static risk factor value is in the range 0-100%, wherein 0% signifies no risk and 100% signifies a very high risk of having a stroke. In another example, static risk factor values in the range of 90-100 correspond to an “extremely high-risk profile” and color coded in red, whereas static risk factor values in the range of 70-90 correspond to a “high risk profile” and color coded in orange. Further, static risk factor values in the range of 50-70 correspond to a “moderate risk profile” and color coded in yellow whereas static risk factor values in the range of 25-50 correspond to a “low risk profile” and color coded in blue. Going further, static risk factor values in the range of 0-25 correspond to “no risk profile” and color coded in green.

In an embodiment, the static risk factor value of the patient may be adjusted manually by a doctor or a caregiver of the patient. In another embodiment, the patient too is enabled to adjust the risk factor value manually. Each static risk profile determines at least one of a frequency of monitoring, requirements for defining a neurological-related event, parameters/settings/configuration/threshold to use for neurological event detection, a diagnostic for validating the neurological-related event, time given for responding to the diagnostic and how soon a designated recipient needs to be alerted on validating the neurological event. Further yet, the designated recipient in the point-of-event diagnostic system is at least one of the group comprising a healthcare provider, a hospital, a health monitoring service, a doctor, a physician, a clinician, a caregiver and a social service.

Further yet, in an embodiment, as mentioned above, a dynamic risk profile may be generated from a second set of parameters obtained from a plurality of mobile communication devices. A dynamic risk factor value is calculated for the patient based on one or more of the dynamic parameters described above in the application.

Typically, the dynamic risk value is in the range of 0-100%, wherein 0% signifies no risk and 100% signifies very high risk for occurrence of stroke-related event. In an example, dynamic risk values in the range of 90-100 correspond to an “extremely high-risk profile” and color coded in red, whereas dynamic risk values in the range of 70-90 correspond to a “elevated risk profile” and color coded in orange. Further, dynamic risk values in the range of 50-70 correspond to a “moderate risk profile” and color coded in yellow, whereas dynamic risk values in the range of 25-50 correspond to a “minimal risk profile” and color coded in blue 25-50. Going further, dynamic risk values in the range of 0-25 correspond to a “no risk profile” stage and color coded in green.

In yet another embodiment of the invention, the point-of-event diagnostic system may comprise of the event-specific physical and, or cognitive diagnostic request be pushed by at least any one of, or a combination of, a manual method, an automatic method and, or a semi-automatic method with or without the aid of a designated recipient. The neurological event is validated by presenting a diagnostic to the patient and analyzing patient's response to the diagnostic.

### Manual Method

In an embodiment, the diagnostic test presented to the patient includes one or more questions and/or instructions. Answers/response given by the patient corresponding to the one or more questions and/or instructions are used to validate the neurological event. The diagnostic may be taken by the patient in assistance with a caregiver located in proximity.

The plurality of questions and/or instructions are used to check the patient's response to at least one of a visual or color appearance, audio or sound and a buzzer or vibration. Further, characteristics of the patient such as slurring of speech, vision impairment and/or fine motor skills may also be checked for the validation of the neurological event. The patient may also be asked to repeat actions, sounds and movements, and answer memory based questions. In an embodiment, the caregiver is also instructed to check a physical characteristic of the patient. Examples of the physical characteristic include, but are not limited to whether the patient is able to raise his arms, facial drooping and paralysis. Typically, the diagnostic presented to the patient and the instructions presented to the caregiver are in accordance with the Face, Arm, Speech, Time (F.A.S.T.) or similar protocol or tests.

### Automatic Method

In another embodiment, the neurological event is primarily validated based on the dynamic risk profile of the patient. In this case, interaction with the patient is not required to validate the neurological event, The dynamic parameters obtained from the a plurality of communication devices being used by the patient are used to validate the stroke-related event in the patient. Additionally, prior history of the patient and other patients in similar conditions is considered to validate the stroke-related event.

### Semi-Automatic Method

In yet another embodiment, both the dynamic risk profile of the patient and the responses of the patient to the diagnostic are considered for validating the neurological event. The neurological event is considered at least one of valid or invalid based on at least one of the validation methods described above. In case, the neurological event is not valid, no action is taken. In case, the neurological event is valid, the designated recipient of the patient is sent an alert. The designated recipient may be in proximity of the patient. In another embodiment, the designated recipient is located at a remote location relative to patient's location. Examples of the alert include, but are not limited to a text message, a phone call, a page and any other communication means thereof.

The designated recipient is presented with a sequence of instructions to confirm the possible onset of a neurological event in the patient. In case, the designated recipient is located remotely, a direct communication link is established between the patient and the designated recipient. Typically, the sequence of instructions is accordance with the Face, Arm, Speech, Time (F.A.S.T.) test protocol. Based on how the patient responds to the F.A.S.T test, the designated recipient calls a medical professional for intervention. An example of such a call is a 911 call. Typically, location of the patient is sent to the medical professional.

Further yet, the point-of-event diagnostic system, may further comprise of a third set of parameters corresponding to an environmental condition surrounding the patient maybe at least one of, wind velocity, temperature, humidity, aridness, light, darkness, noise pollution, exposure to UV, airborne pollution and radioactivity.

Further yet, additionally, in an embodiment of the invention, the point-of-event diagnostic system may deduct a threshold discrepancy of the neurological event type using machine learning algorithm.

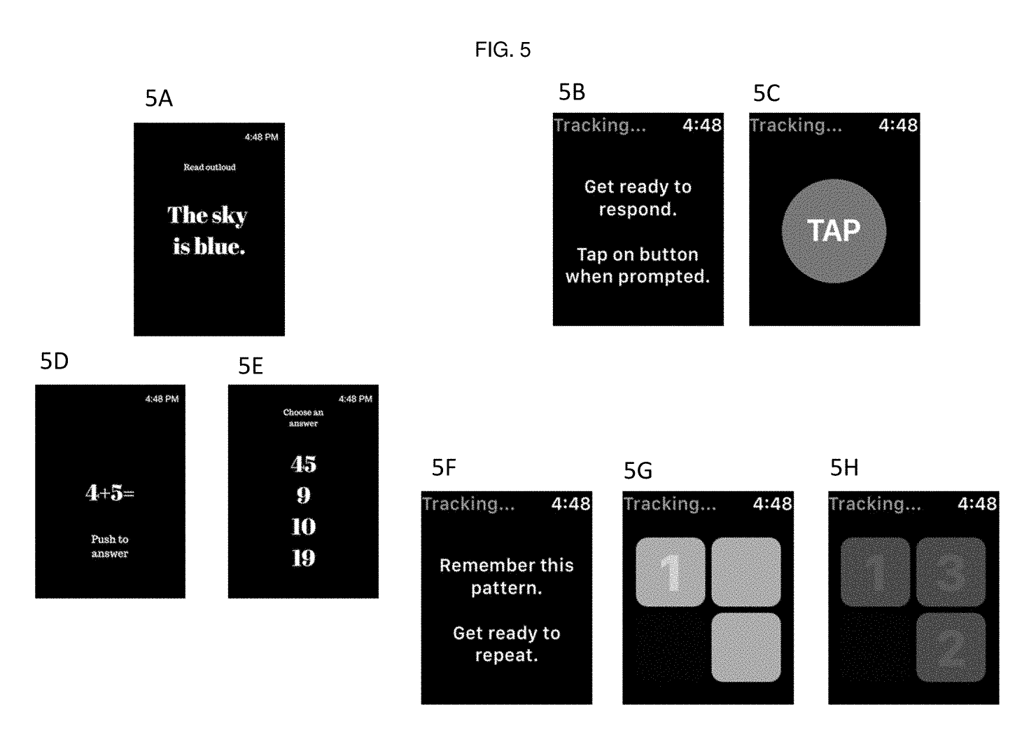

FIG. 5 show a user interface according to an exemplary embodiment of the invention. As mention above, detection of a threshold-grade deviation deduces a neurological event type and based on the neurological event type, an event-specific physical or cognitive diagnostic is pushed to the patient. Responsiveness assessments can be incorporated on to the plurality of body-worn devices or any other communication devices to assess at least one of, but not limited to, the patients' responsiveness, awareness, alertness, grogginess and or cognition.

Further yet, in an embodiment of the invention, the reconfigurable point-of-event diagnostic system comprise of the event-specific physical and, or cognitive diagnostic to be dynamic wherein, the event-specific diagnostic presented to the patient may be at least one of, in increasing difficulty when the patient responds correctly, in decreasing difficulty when the patient responds incorrectly to the diagnostic and, or a variable combination of increasing or decreasing difficulty. Additionally, in an embodiment the event-specific diagnostic given to the patient maybe at least one of, a visual or color appearance, audio or sound, buzzer, vibration, fine motor skills, memory based tasks, repeating actions, sounds and, or movements.

Below are some examples of the diagnostic presented to the patient/user. Here's an example of a physical diagnostic. A body-worn device will generate a signal (i.e., 1, 2, or 3 pulses) to which the subject would respond by pressing a watch button the same number of times. This test will assess fine motor response by the ability to push buttons on the wearable device or interacting with the touch screen on any other communication device. It will also assess subject's awareness of the haptic input (gentle vibration or buzz) provided by the wearable device. Additionally, we present an example of a cognitive diagnostic as shown in FIG. 5A, where a wearable device will generate a language task by alerting the subject to read a text message on the watch aloud and capture audio. The audio will help evaluate slurred speech, blurred vision and general awareness and ability to focus and read small print. FIG. 5B, 5C show responsiveness of the patient by presenting instructions like “tap on button when prompted”. Further yet, as shown in FIG. 5E, 5D, a wearable device will test attention and calculation by generating simple math problems (i.e., 1+2) and having the subject answer with the correct number of button pushes. Additionally, FIGS. 5F, 5G and 5H show when the patient will be presented with a simple pattern on a grid and asked to recreate the pattern on the screen either by pushing a button on the wearable device (where available and applicable) or by interacting with the device's touch screen.

These diagnostics can be presented in the order of increasing difficulty. Alternatively, the diagnostic may be present in at least one or combination of, but not limited to, increasing or decreasing difficulty, gradient of increasing or decreasing difficulty, wave pattern of difficulty. The patient must complete the simpler ones successfully before the more difficult ones are presented. This allows accurate evaluation of the subject's awareness, responsiveness and cognition. These tests can be used to evaluate “Before”, “After” episodic neurological events. This capability will provide an entirely new way of assessing post-episode recovery for a variety of acute conditions such as seizures, stroke, heart attacks and progression of disease in the patient for conditions such as Alzheimer's, MS and more. It also provides a non-invasive way of conducting periodic assessments of cognitive function in those with degenerative conditions such as Alzheimer's, Multiple Sclerosis etc.

FIG. 6. depicts a method flowchart for validation of neurological events. In an exemplary embodiment of the invention, a reconfigurable point-of-event diagnostic method comprises the steps of (1) capturing at least a set of physiological, physical and, or motion parameter of a patient 602; (2) generating a static risk profile, and detecting a threshold-grade deviation therefrom to deduce a neurological event type based on a set of parameters 603; (3) pushing at least one of an event-specific cognitive and, or physical diagnostic based on the neurological event type 604; (5) validating the neurological event by the validation module based on the response to at least one event-specific cognitive and, or physical diagnostic 605; and (6) pushing information of the validated neurological event to any one of a designated recipient 606.

Further yet, in an embodiment of the invention, the method of reconfigurable point-of-event diagnostic wherein, the static risk profile is probabilistic-learning enabled to update the patient's personal static risk profile based on at least one of, the detected physiological, physical, and, or motion parameters of the patient, and, or the validated neurological event based on diagnostic responses by the patient. Further yet, the validation module is probabilistic-learning enabled to update a validation table based on at least one of a checklist of detected parameters and patient responses to the pushed diagnostic.

In yet another embodiment of the invention, a reconfigurable point-of-event diagnostic device may comprise of at least one embedded sensor configured for capturing at least one of a physical, physiological and, or motion parameter, a network interface operably coupling the device to a mobile device with a wireless network access. Further yet, the device is configured to capture at least one of the physiological, physical and, or motion parameters of a patient and compare to a static risk profile and detect a threshold-grade deviation therefrom to deduce a neurological event type, based on the neurological event type, push at least one of an event-specific cognitive and, or physical diagnostic, based on the response to at least one event-specific cognitive and, or physical diagnostic, validate the neurological event by the validation module and push an informational of the validated neurological event to any one of a designated recipient.

Further yet, in an embodiment of the invention, the device is further configured to capture a second set of at least one of a physiological, physical and, or motion parameter from a second device on the patient. Additionally, the device is further configured to capture a third set of an environmental parameter related to the patient from a third input source remote of the patient.

In another embodiment of the invention, the informational pushed to the patient's device is a single or sequence of non-verbal and non-textual cues coding a pre-defined message. Moreover, the single or sequence of non-verbal and non-textual cues is at least one of a static and, or patterned flashing light of varying colors on a display and, or surround of the device coding a pre-defined message. Further yet, the single or sequence of non-verbal and non-textual cues is at least one of a static and, or patterned haptic from the device coding a pre-defined message.

Additionally, a set of pre-defined message codes for varying levels of urgency, reducing response times. For example, red might indicate an imminent urgency, whereas orange might indicate low urgency. Further yet, in an embodiment, a validated level of urgency is expressed in a text-based informational and pushed to at least one of a designated recipient, reducing response times.

Embodiments are described at least in part herein with reference to flowchart illustrations and/or block diagrams of methods, systems, and computer program products and data structures according to embodiments of the disclosure. It will be understood that each block of the illustrations, and combinations of blocks, can be implemented by computer program instructions. These computer program instructions may be provided to a processor of a general-purpose computer, special purpose computer, or other programmable data processing apparatus to produce a machine such that the instructions, which execute via the processor of the computer or other programmable data processing apparatus, create means for implementing the functions/acts specified in the block or blocks.

These computer program instructions may also be stored in a computer-readable memory that can direct a computer or other programmable data processing apparatus to function in a particular manner such that the instructions stored in the computer-readable memory produce an article of manufacture including instruction means which implement the function/act specified in the block or blocks.

The computer program instructions may also be loaded onto a computer or other programmable data processing apparatus to cause a series of operational steps to be performed on the computer or other programmable apparatus, to produce a computer implemented process such that, the instructions which execute on the computer or other programmable apparatus provide steps for implementing the functions/acts specified in the block or blocks.

In general, the word “module” as used herein, refers to logic embodied in hardware or firmware, or to a collection of software instructions, written in a programming language, such as, Java, C, etc. One or more software instructions in the unit may be embedded in firmware. The modules described herein may be implemented as either software and/or hardware modules and may be stored in any type of non-transitory computer-readable medium or other non-transitory storage elements. Some non-limiting examples of non-transitory computer-readable media include CDs, DVDs, BLU-RAY, flash memory, and hard disk drives.

In the drawings and specification, there have been disclosed exemplary embodiments of the disclosure. Although specific terms are employed, they are used in a generic and descriptive sense only and not for purposes of limitation, the scope of the invention being defined by the following claims. Those skilled in the art will recognize that the present invention admits of a number of modifications, within the spirit and scope of the inventive concepts, and that it may be applied in numerous applications, only some of which have been described herein. It is intended by the following claims to claim all such modifications and variations which fall within the true scope of the invention.

1. A reconfigurable point-of-event diagnostic system comprising:
a body-worn device configured for capturing at least one of a physical, physiological and, or motion parameter;
a network interface;
a processor;
a validation module;
a non-transitory storage element coupled to the processor;
encoded instructions stored in the non-transitory storage element, wherein the encoded instructions when implemented by the processor, configure the diagnostic system to:
capture at least one of the physiological, physical and, or motion parameter of a patient, compare to a static risk profile and detect a threshold-grade deviation therefrom to deduce a neurological event type;
based on the neurological event type, push at least one of an event-specific cognitive and, or physical diagnostic;
based on the response to at least one event-specific cognitive and, or physical diagnostic, validate the neurological event by the validation module; and
push an information of the validated neurological event to any one of a designated recipient.

2. The point-of-event diagnostic system of claim 1, wherein the motion characteristics of the patient correspond to at least one of activity related characteristics or sleep related characteristics of the patient.

3. The point-of-event diagnostic system of claim 1, wherein the set of parameters corresponding to the activity related characteristics of the patient is at least one of the group comprising maximum value of acceleration, minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

4. The point-of-event diagnostic system of claim 1, wherein the set of parameters corresponding to the sleep related characteristics of the patient is indicative of at least one of the group comprising sleep time, number of times awake, duration of sound sleep, duration of light sleep and awake time.

5. The point-of-event diagnostic system of claim 1, wherein the set of parameters corresponding to the motion characteristics of the patient are captured by one or more sensors selected from the group comprising a motion sensor, an accelerometer, a 3D accelerometer, a gyroscope, a global positioning system sensor (GPS), a magnetometer, an inclinometer and an impact sensor.

6. The point-of-event diagnostic system of claim 1, wherein a set of parameters corresponding to physiological characteristics of the patient is at least one of group comprising heart rate, pulse rate, respiratory rate and body temperature.

7. The point-of-event diagnostic system of claim 1, further comprising a second set of parameters is provided by the patient using at least one of a mobile communication device and, or any other device.

8. The point-of-event diagnostic system of claim 1, wherein a set of parameters corresponding to the physical characteristics is based on at least one of, age, gender, race, medical history, heart rate, medication history, blood pressure, sweat, duration, number of episodes and history of disease, family history, fatigue, walking/running/movement related impairment, bladder dysfunction, vision and speech impairments, food habits, smoking, alcohol and drug intake.

9. The point-of-event diagnostic system of claim 1, comprising constructing static risk profile based on at least one of, the set of physical, physiological or motion parameters and, or a plurality of combinations from a finite set of the set of parameters.

10. The point-of-event diagnostic system of claim 1, further comprising establishing a personal static risk profile of the patient based on at least one of the set of parameters.

11. The point-of-event diagnostic system of claim 1, further comprising detecting a deviation from the personal static risk profile of the patient using machine learning algorithm.

12. The point-of-event diagnostic system of claim 1, further comprising the event-specific physical and, or cognitive diagnostic request may be pushed by at least any one of, or a combination of, a manual method, an automatic method and, or a semi-automatic method with or without the aid of a designated recipient.

13. A reconfigurable point-of-event diagnostic system of claim 1, further comprising the event-specific physical and, or cognitive diagnostic to be dynamic wherein, the event-specific diagnostic presented to the patient may be at least one of, in increasing difficulty when the patient responds correctly, in decreasing difficulty when the patient responds incorrectly to the diagnostic and, or a variable combination of increasing or decreasing difficulty.

14. The point-of-event diagnostic system of claim 1, wherein the event-specific physical and, or cognitive diagnostic may be validated by the validation module by presenting a event-specific diagnostic to the patient and analyzing the patient's response to the event-specific diagnostic.

15. The point-of-event diagnostic system of claim 1, wherein the event-specific diagnostic given to the patient maybe at least one of, a visual or color appearance, audio or sound, buzzer, vibration, fine motor skills, memory based tasks, repeating actions, sounds and, or movements.

16. The point-of-event diagnostic system of claim 1, wherein the event-specific physical and, or cognitive diagnostic may be validated by the validation module obtaining data from any one of, body worn sensors, sensor based devices used by patients, mobile communication device, smartphone, tablet, personal digital assistant (PDA) and, or Internet of Things device.

17. The point-of-event diagnostic system of claim 1, further comprising, a third set of parameters corresponding to an environmental condition surrounding the patient maybe at least one of, wind velocity, temperature, humidity, aridness, light, darkness, noise pollution, exposure to UV, airborne pollution and radioactivity.

18. The point-of-event diagnostic system of claim 1, wherein machine learning algorithm may deduct a threshold discrepancy of the neurological event type.

19. The point-of-event diagnostic system of claim 1, wherein aggregation of the set of parameters occurs from at least one of, a mobile communication device, wearable device, smartphone, tablet, personal digital assistant (PDA) and Internet of Things device.

20. The point-of-event diagnostic system of claim 1, wherein the designated recipient is at least one of the group comprising a healthcare provider, a hospital, a health monitoring service, a doctor, a physician, a clinician, a caregiver and a social service.

21. A reconfigurable point-of-event diagnostic system of claim 1, further comprising integration with any one of a third-party application via an Application Program Interface (API).

22. A reconfigurable point-of-event diagnostic system of claim 1, further comprising integration with any one of, electronic medical records (EMR), remote server, and, or a cloud-based server for down-stream analytics and, or provisioning.

23. A reconfigurable point-of-event diagnostic system of claim 1, wherein at least one event-specific diagnostic triggers at least one action controlled by a “if this, then that” script manager.

24. A reconfigurable point-of-event diagnostic system of claim 1, wherein a “if this, then that” script manager is further embedded with an “and, or” trigger or action operators, allowing increased triggers or actions in a command set.

25. A reconfigurable point-of-event diagnostic system of claim 1, wherein the patient alert is at least one of, text, email, vibration with or without audible notification, visual display, and, or a color-coded or blinking notification.

26. A reconfigurable point-of-event diagnostic method comprising:
capturing at least a set of physiological, physical and, or motion parameter of a patient;
generating a static risk profile, and detecting a threshold-grade deviation therefrom to deduce a neurological event type based on a set of parameters;
pushing at least one of an event-specific cognitive and, or physical diagnostic based on the neurological event type;
validating the neurological event by the validation module based on the response to at least one event-specific cognitive and, or physical diagnostic; and
pushing information of the validated neurological event to any one of a designated recipient.

27. The method of claim 26, wherein the static risk profile is probabilistic-learning enabled to update the patient's personal static risk profile based on at least one of, the detected physiological, physical, and, or motion parameters of the patient, and, or the validated neurological event based on diagnostic responses by the patient.

28. The method of claim 26, wherein the validation module is probabilistic-learning enabled to update a validation table based on at least one of a checklist of detected parameters and patient responses to the pushed diagnostic.

29. A reconfigurable point-of-event diagnostic device comprising:
at least one embedded sensor configured for capturing at least one of a physical, physiological and, or motion parameter;
a network interface operably coupling the device to a mobile device with a wireless network access, wherein the device is configured to:
capture at least one of the physiological, physical and, or motion parameters of a patient and compare to a static risk profile and detect a threshold-grade deviation therefrom to deduce a neurological event type;
based on the neurological event type, push at least one of an event-specific cognitive and, or physical diagnostic;
based on the response to at least one event-specific cognitive and, or physical diagnostic, validate the neurological event by the validation module; and
push an informational of the validated neurological event to any one of a designated recipient.

30. The diagnostic device of claim 29, wherein the device is further configured to capture a second set of at least one of a physiological, physical and, or motion parameter from a second device on the patient.

31. The diagnostic device of claim 29, wherein the device is further configured to capture a third set of an environmental parameter related to the patient from a third input source remote of the patient.

32. The diagnostic device of claim 29, wherein the informational pushed to the patient's device is a single or sequence of non-verbal and non-textual cues coding a pre-defined message.

33. The diagnostic device of claim 29, wherein the single or sequence of non-verbal and non-textual cues is at least one of a static and, or patterned flashing light of varying colors on a display and, or surround of the device coding a pre-defined message.

34. The diagnostic device of claim 29, wherein the single or sequence of non-verbal and non-textual cues is at least one of a static and, or patterned haptic from the device coding a pre-defined message.

35. The diagnostic device of claim 29, wherein the pre-defined message codes for varying levels of urgency, reducing response times.

36. The diagnostic device of claim 35, wherein a validated level of urgency is expressed in a text-based informational and pushed to at least one of a designated recipient, reducing response times.
